(19) United States
(12) Patent Application Publication
(10) Pub. No.: US 2019/0328976 A1
(43) Pub. Date: Oct. 31, 2019

(54) COMBINATION ACCESSORY FINGER FLANGE AND SYRINGE WITH A NEEDLE-STICK PREVENTION DEVICE

(71) Applicant: TECH GROUP EUROPE LIMITED, Dublin 2 (IE)

(72) Inventors: Christopher EVANS, Long Valley, NJ (US); Brian COSTELLO, Union City, NJ (US)

(73) Assignee: Tech Group Europe Limited, Dublin 2 (IE)

(21) Appl. No.: 16/312,425

(22) PCT Filed: Jun. 14, 2017
(86) PCT No.: PCT/IB2017/000881

§ 371 Date: Dec. 21, 2018

Related U.S. Application Data
(60) Provisional application No. 62/356,658, filed on Jun. 30, 2016.

(51) Int. Cl. A61M 5/31

(52) U.S. Cl. CPC A61M 5/3137; A61M 5/3243; A61M 5/3135; A61M 2005/3139

(57) ABSTRACT

A combination accessory finger flange and pharmaceutical syringe system including a needle safety shield having a safety-shield outer tube the proximal end of which forms a coupling ring, has a base portion with diametrically-opposed finger flanges that extend radially outwardly therefrom and a central opening dimensioned to receive the safety-shield outer tube. Diametrically-opposed, radially outwardly extending coupling-ring flanges are received and retained in diametrically-opposed receptacles on a top surface of the base-portion adjacent the central opening when the safety-shield outer tube is in the central opening, the coupling-ring flanges are in contact with the top surface of the base-portion and the coupling ring is rotated in a first rotational direction relative to the base portion.

### BACKGROUND OF THE INVENTION

The present invention relates to a combination accessory finger flange and pharmaceutical syringe. More particularly, the present invention relates to an accessory finger flange attachable to a syringe with a needle-stick prevention device having small molded flanges that are inadequate in size, shape and ergonomics and, therefore, unable to allow a user to have confident control over the position of the syringe during administration of an intradermal medicament.

Conventional needle-stick prevention devices intended to reduce the frequency of post-injection needle-sticks typically have a sheath partially or completely surrounding the syringe. The sheath may be held in a retracted position exposing the needle for aspiration and injection and may be automatically deployed around a needle afterwards.

A common form of safety injection device for a syringe completely surrounds the syringe and can be automatically activated. U.S. Pat. No. 9,022,990, entitled “Needle Safety Shield”, incorporated herein by reference, discloses a pharmaceutical syringe system 1 including a needle safety shield 2 having a safety-shield outer tube 3 with a syringe barrel (not shown) disposed therein (see FIG. 5). The proximal end 3a of the safety-shield outer tube 3 forms a coupling ring 4 connecting the proximal end of the syringe barrel to the safety-shield outer tube 3. The coupling ring 4 has diametrically-opposed, radially outwardly extending coupling-ring flanges 5. The coupling ring 4 completely encloses the finger flange (not shown) at the proximal end of the syringe barrel. Progressive axial displacement of the actuation head 6 releases a latching mechanism in the coupling ring 4 allowing an inner tube 7 in the outer tube 3 to extend to cover the cannula 8. The coupling flanges 5 are inadequate in size, shape and ergonomics to enable a user to have confident control over the position of the cannula 8 during administration of an intradermal medicament.

Accordingly, there is a need for an accessory finger flange to improve the ergonomics of pharmaceutical syringes with a needle-stick prevention device with flanges that are inadequate in size, shape and ergonomics to effectively function as finger flanges.

### BRIEF SUMMARY OF THE INVENTION

Briefly stated, one embodiment of the present invention is directed to a combination accessory finger flange and pharmaceutical syringe system, including a needle safety shield having a safety-shield outer tube with a syringe barrel disposed therein. A proximal end of the safety-shield outer tube forms a coupling ring connecting the proximal end of the syringe barrel to the safety-shield outer tube. The coupling ring has diametrically-opposed, radially outwardly extending coupling-ring flanges. An accessory finger flange including a base portion having a central opening receives the safety-shield outer tube therein. The base portion includes a base-portion top surface, a base-portion bottom surface, and diametrically-opposed receptacles positioned on the base-portion top surface adjacent the central opening. The receptacles receive and retain the coupling-ring flanges therein such that the coupling-ring flanges are in contact with the base-portion top surface and the coupling ring is rotated in a first rotational direction relative to the base portion until the coupling-ring flanges are received in the receptacles.

### BRIEF DESCRIPTION OF THE SEVERAL VIEWS OF THE DRAWINGS

The foregoing summary, as well as the following detailed description of a preferred embodiment of the invention, will be better understood when read in conjunction with the appended drawings. For the purpose of illustrating the invention, the drawings show an embodiment which is presently preferred. It should be understood, however, that the invention is not limited to the precise arrangements and instrumentalities shown.

In the drawings:

FIG. 1 is a front elevation view of a first preferred embodiment of an accessory finger flange in accordance with the present invention;

FIG. 2 is a top plan view of the accessory finger flange of FIG. 1;

FIG. 3 is a top perspective view of the accessory finger flange of FIG. 1;

FIG. 4 is a front elevation view of the accessory finger flange of FIG. 1 mounted on a pharmaceutical syringe system; and

FIG. 5 is a front elevation view of a prior art syringe system with a needle-stick prevention device.

### DETAILED DESCRIPTION OF THE INVENTION

Reference will now be made in detail to an embodiment of the invention, examples of which are illustrated in the accompanying drawings. The terminology used in the description of the invention herein is for the purpose of describing a particular embodiment only and is not intended to be limiting of the invention.

As used in the description of the invention and the appended claims, the singular forms “a”, “an” and “the” are intended to include the plural forms as well, unless the context clearly indicates otherwise. The words “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The words “top”, “bottom’ and “proximal” designate directions in the drawings to which reference is made. The words “inwardly” and “outwardly” refer to directions toward and away from, respectively, the geometric center of the needle safety shield, and designated parts thereof. The terminology includes the words noted above, derivatives thereof and words of similar import.

The following description is directed towards a preferred embodiment of an accessory finger flange in accordance with the present invention.

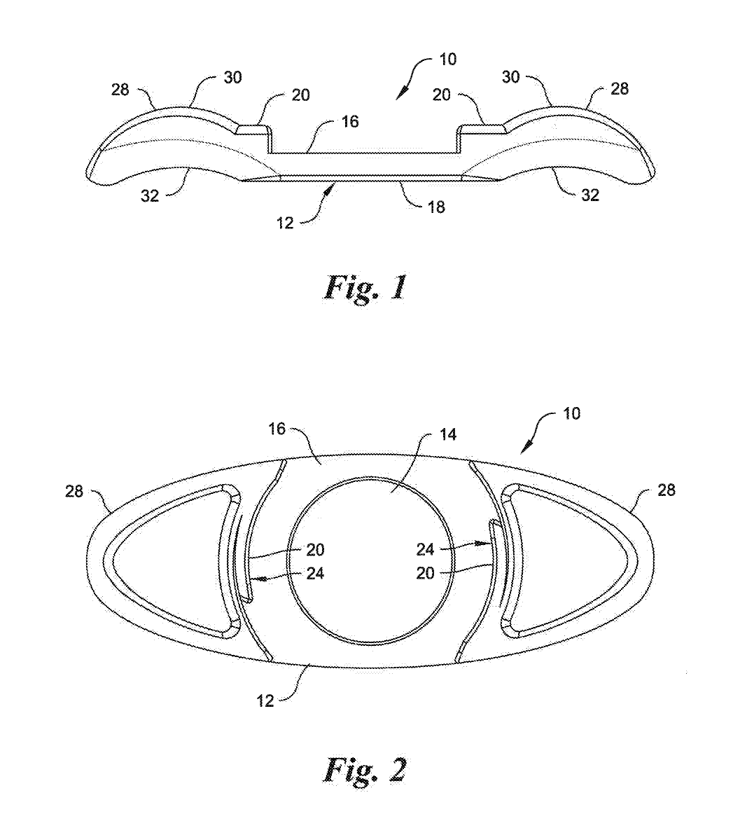

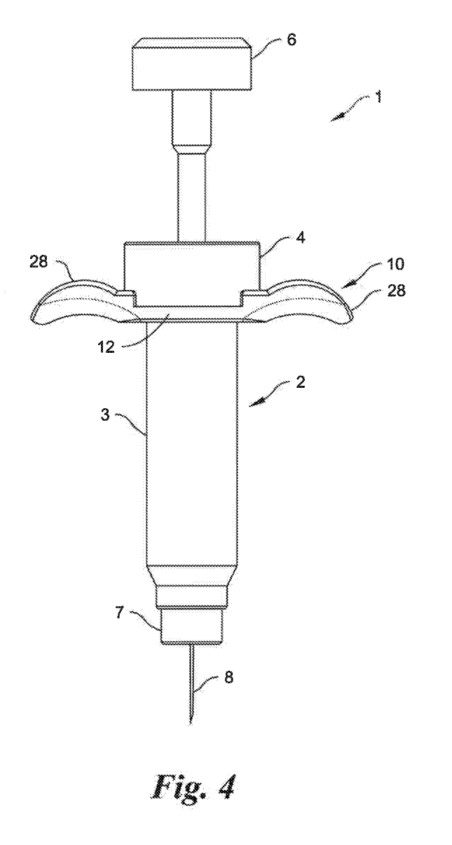

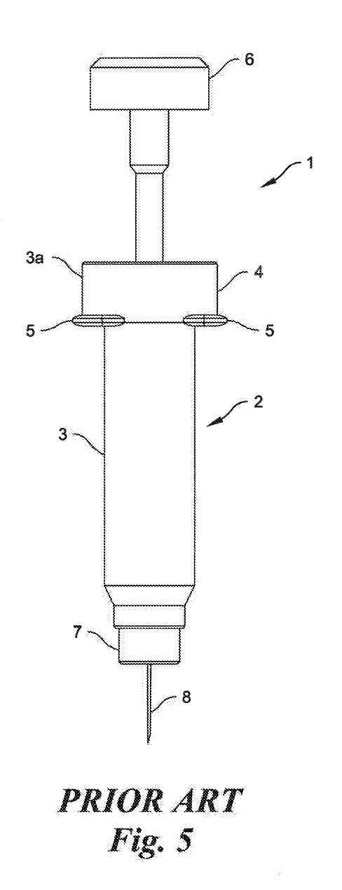

Referring to the drawings in detail, where like numerals indicate like elements throughout, there is shown in FIGS. 1-4 a first preferred embodiment of an accessory finger flange, generally designated 10, and hereinafter referred to as the “accessory flange” 10 in accordance with the present invention. The accessory flange 10 is for use with a pharmaceutical syringe system 1 including a needle safety shield 2 having a safety-shield outer tube 3 with a syringe barrel (not shown) disposed therein (see FIG. 5). The proximal end 3a of the safety-shield outer tube 3 forms a coupling ring 4 connecting the proximal end of the syringe barrel to the safety-shield outer tube 3. The coupling ring 4 has diametrically-opposed, radially outwardly extending coupling-ring flanges 5.

The accessory finger flange 10 comprises a base portion 12 having a central opening 14 dimensioned to receive the safety-shield outer tube 3 of the syringe system 1. The base portion 12 includes a base-portion top surface 16 and a base-portion bottom surface 18. Preferably, the base portion 12 is generally linear in cross-sectional shape.

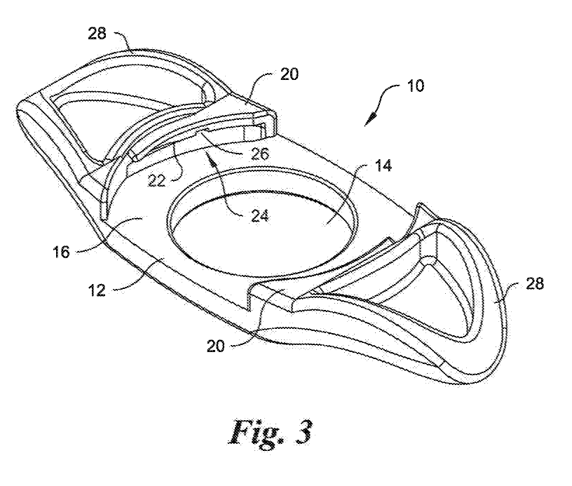

Diametrically-opposed receptacles 20 are on the base-portion top surface 16 adjacent the central opening 14. The receptacles 20 are configured to receive and retain the coupling-ring flanges 5. Preferably, each of the diametrically-opposed receptacles 20 has a cammed surface 22 (see, FIG. 3) spaced from and facing an opposing base-portion top surface 24. The cammed surface 22 is angled downwardly toward the opposing base-portion top surface 24.

The coupling-ring flanges 5 are received and retained in the diametrically-opposed receptacles 20 when the safety-shield outer tube 3 is in the central opening 14, the coupling-ring flanges 5 are in contact with the base-portion top surface 16 and the coupling ring 4 is rotated in a first rotational direction relative to the base portion 12 until the coupling-ring flanges 5 are received in the receptacles 20. The range of rotation can be suggestedly at least about ten degrees, desirably twenty or more degrees, preferably between about forty-five to ninety degrees and less preferably no more than 120 degrees. In some embodiments, the retention may be irreversible; in other embodiments, rotation in a second rotational direction opposite the first rotational direction may decouple the accessory finger flange 10 from the syringe system 1.

Preferably, the coupling-ring flanges 5 are retained in the receptacles 20 by a friction fit between the cammed surface 22 and the opposing base-portion top surface 24. In some embodiments, the cammed surface 22 has a detent 26 (see, FIG. 3) and audible and tactile signals are produced when the coupling-ring flanges 5 first contact the detents 26.

Diametrically-opposed finger flanges 28 extend radially outwardly from the base portion 12. Each diametrically-opposed finger flange 28 has a top finger-flange surface 30 and a bottom finger-flange surface 32. The top and bottom finger flange surfaces 30, 32 are preferably nonlinear in cross-sectional shape. In some embodiments, the nonlinear in cross-sectional shape of the top finger-flange surface 30 is a convex shape and the nonlinear in cross-sectional shape of the bottom finger-flange surface 32 is a concave shape.

The accessory finger flange 10 is intended for use with a pharmaceutical syringe, such as the syringe 1 shown in FIG. 5, with a needle-stick prevention device with flanges that are inadequate in size, shape and ergonomics to effectively function as finger flanges. To allow a user to have confident control over the positioning of the syringe during administration of an intradermal medicament, the safety-shield outer tube 3 is inserted in the central opening 14 of the base portion 12 of the accessory finger flange 10 until the coupling-ring flanges 5 are in contact with the base-portion top surface 16. Then, the coupling ring 4 is fixedly attached to the accessory finger flange 10 by rotating the coupling ring 4 in a first rotational direction relative to the base portion 12 until the coupling-ring flanges 5 are received and retained in the diametrically-opposed coupling-ring receptacles 20 on the base-portion top surface 16 adjacent the central opening 14.

Although the foregoing embodiment of the accessory finger flange 10 has been disclosed for use with a syringe with a particular needle-stick prevention device, the accessory finger flange 10 may be used with syringes with other needle-stick prevention devices and may also be used with syringes without a needle-stick prevention device. An artisan will understand that the diametrically-opposed receptacles on the base portion 12 of the accessory finger flange 10 may be reconfigured to receive and retain structures extending radially outwardly from the outermost surface of a needle-stick prevention device or syringe and having a wide variety of geometrical cross sectional shapes.

The foregoing detailed description of the invention has been disclosed with reference to a specific embodiment. However, the disclosure is not intended to be exhaustive or to limit the invention to the precise form disclosed. Those skilled in the art will appreciate that changes could be made to the embodiment described above without departing from the broad inventive concept thereof. Therefore, the disclosure is intended to cover modifications within the spirit and scope of the present invention as defined by the appended claims.

1-5. (canceled)

6. A combination accessory finger flange and pharmaceutical syringe system comprising:
a needle safety shield having a safety-shield outer tube with a syringe barrel disposed therein, a proximal end of the safety-shield outer tube forming a coupling ring connecting the proximal end of the syringe barrel to the safety-shield outer tube, the coupling ring having diametrically-opposed, radially outwardly extending coupling-ring flanges;
an accessory finger flange including a base portion having a central opening dimensioned to receive the safety-shield outer tube therein, the base portion including a base-portion top surface and a base-portion bottom surface, diametrically-opposed receptacles positioned on the base-portion top surface adjacent the central opening, the receptacles receiving and retaining the coupling-ring flanges therein such that in a first position the coupling-ring flanges are in contact with the base-portion top surface and extend beyond an edge of the top surface and in a second position such that when the coupling ring is rotated in a first rotational direction relative to the base portion, the coupling-ring flanges are received in the receptacles and do not extend beyond an edge of the top surface.

7. The accessory finger flange according to claim 1, wherein each of the diametrically-opposed receptacles has a cammed surface spaced from and facing an opposing base-portion top surface, the cammed surface angled downwardly toward the opposing base-portion top surface and the coupling-ring flanges are retained in the receptacles by a friction fit between the cammed surface and the opposing base-portion top surface.

8. The accessory finger flange according to claim 2, wherein each cammed surface has a detent and audible and tactile signals are produced when the coupling-ring flanges first contact the detents.

9. The accessory finger flange according to claim 1, wherein the diametrically-opposed finger flanges have a top finger-flange surface and a bottom finger-flange surface, each surface being nonlinear in cross-sectional shape and the base portion is linear in cross-sectional shape.

10. The accessory finger flange according to claim 4, wherein the nonlinear in cross-sectional shape of the top finger-flange surface is a convex shape and the nonlinear in cross-sectional shape of the bottom finger-flange surface is a concave shape.
